(19) United States
(12) Patent Application Publication
(10) Pub. No.: US 2024/0034996 A1
(43) Pub. Date: Feb. 1, 2024

(54) GENE MODIFIED IPSC DERIVED CELLULAR COMPOSITIONS FOR REGENERATION AND IMMUNE MODULATION

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); Famela RAMOS, Oceanside, CA (US); James VELTMEYER, Oceanside, CA (US)

(73) Assignee: Therapeutic Solutions International, Inc., Oceanside, CA (US)

(21) Appl. No.: 18/361,712

(22) Filed: Jul. 28, 2023

Related U.S. Application Data
(60) Provisional application No. 63/393,451, filed on Jul. 29, 2022.

(51) Int. Cl. C12N 5/0784; C12N 5/0775

(52) U.S. Cl. CPC C12N 5/0639; C12N 5/0662; C12N 2506/45; C12N 2501/60; C12N 2501/603; C12N 2501/602; C12N 2501/605; C12N 2501/73

(57) ABSTRACT

Disclosed are cells and cellular compositions useful for treatment of degenerative and/or autoimmune diseases derived from gene edited/gene modified pluripotent stem cells. In one embodiment pluripotent stem cell such as inducible pluripotent stem cells are gene modified to express tissue associated transcription factors such as pdx-1 if endodermal tissue is desired and cells are differentiated into regenerative-type cells such as along the mesenchymal lineage. In one embodiment the invention teaches transfection with IL-27 to induce expression of coinhibitory molecules for suppression of autoimmunity. In some embodiments the invention provides generation of iPSC derived MSC which can not stimulate inflammation due to gene-editing based removal of inflammatory associated transcription factors.

### CROSS-REFERENCE TO RELATED APPLICATIONS

This application claims priority to U.S. Provisional Application No. 63/393,451, filed Jul. 29, 2022, entitled “Gene Modified iPSC Derived Cellular Compositions for Regeneration and Immune Modulation”, which is incorporated by reference herein in its entirety.

### FIELD OF THE INVENTION

The teachings herein relate to the use of modified stem cells for the use in regenerative medicine and immune modulation

### BACKGROUND OF THE INVENTION

There is a need in the art for development of more potent stem cell therapeutics. To date, no stem cell therapy has been demonstrated to reproducibly benefit in phase III trials with exception of graft versus host disease. The invention provides means of creating stem cells capable of inducing tissue repair and concurrent immune modulation.

### SUMMARY

Preferred embodiments are directed to methods of treating a degenerative condition comprising the steps of: a) obtaining a pluripotent stem cell; b) inducing expression in said pluripotent stem cell of one or more factors capable of promoting differentiation into desired cell lineage; c) differentiating said stem cell into a regenerative cell; d) optionally endowing said differentiated regenerative cell with chemoattractant activity; and e) optionally endowing said regenerative cell with immune modulatory activity.

Preferred methods include embodiments wherein said degenerative condition is associated with loss of function of one more organ systems.

Preferred methods include embodiments wherein said degenerative condition is associated with deterioration of function of one more organ systems.

Preferred methods include embodiments wherein said degenerative condition is associated with fibrosis one more organ systems.

Preferred methods include embodiments wherein said degenerative condition is associated with inflammation occurring in one more organ systems.

Preferred methods include embodiments wherein said degenerative condition is associated with autoimmune attack against one more organ systems.

Preferred methods include embodiments wherein said degenerative condition is associated with denervation of one more organ systems.

Preferred methods include embodiments wherein said degenerative condition is associated with fibrosis one more organ systems.

Preferred methods include embodiments wherein said organ systems are chosen from a group comprising of: a) respiratory system; b) digestive and excretory system; c) circulatory system; d) urinary system; e) integumentary system; f) skeletal system; g) muscular system h) endocrine system; i) lymphatic system; j) nervous system; and k) reproductive systems.

Preferred methods include embodiments wherein said pluripotent stem cell is an inducible pluripotent stem cell.

Preferred methods include embodiments wherein said pluripotent stem cell is generated by dedifferentiation.

Preferred methods include embodiments wherein said dedifferentiation is accomplished by introduction into cells proteins capable of inducing dedifferentiation.

Preferred methods include embodiments wherein said dedifferentiation results in cells expression pluripotency markers.

Preferred methods include embodiments wherein said pluripotency marker is TRA-1-60.

Preferred methods include embodiments wherein said pluripotency marker is hTERT.

Preferred methods include embodiments wherein said pluripotency marker is TRA-1-81

Preferred methods include embodiments wherein said pluripotency marker is SSEA4.

Preferred methods include embodiments wherein said pluripotency marker is OCT4.

Preferred methods include embodiments wherein said pluripotency marker is SOX2.

Preferred methods include embodiments wherein said proteins capable of inducing dedifferentiation are selected from a group comprising of: a) OCT4; b) NANOG; c) KLF-1; d) SOX-2; and e) h-RAS.

Preferred methods include embodiments wherein mRNA is introduced into said cells in order to induce expression of pluripotency inducing genes.

Preferred methods include embodiments wherein said dedifferentiated cells are capable of proliferating for more than 50 passages.

Preferred methods include embodiments wherein MSC are generated from pluripotent stem cells.

Preferred methods include embodiments wherein said MSC are differentiated by exposure to BMP-2.

Preferred methods include embodiments wherein said MSC are differentiated by exposure to BMP-2 and FGF-1.

Preferred methods include embodiments wherein said MSC are differentiated by exposure to FGF-1.

Preferred methods include embodiments wherein said MSC are differentiated by exposure to bone marrow stroma.

Preferred methods include embodiments wherein said MSC are differentiated by exposure to placental stroma.

Preferred methods include embodiments wherein said MSC are differentiated by exposure to thymic stroma.

Preferred methods include embodiments wherein said MSC are differentiated by exposure to ovarian stroma.

Preferred methods include embodiments wherein said MSC are differentiated by exposure to submucosal intestinal epithelial cell stroma.

Preferred methods include embodiments wherein said MSC are generated from pluripotent stem cells transfected with transcription factors promoting tissue specific differentiation.

Preferred methods include embodiments wherein said MSC are transfected with IL-27 to induce anti-inflammatory effects.

Preferred methods include embodiments wherein said MSC are utilized for treatment of autoimmunity.

Preferred methods include embodiments wherein pluripotent stem cells are transfected with PDX-1 when differentiated MSC are used to treat liver failure.

Preferred methods include embodiments wherein said MSC are activated with an mimic of an injury signal to endow enhanced regenerative activity from said MSC.

Preferred methods include embodiments wherein said mimic of an injury signal is lipopolysaccharide.

Preferred methods include embodiments wherein said mimic of an injury signal is poly (IC).

Preferred methods include embodiments wherein said mimic of an injury signal is free histones.

Preferred methods include embodiments wherein said mimic of an injury signal is CpG motifs.

Preferred methods include embodiments wherein said mimic of an injury signal is imiquimod.

Preferred methods include embodiments wherein said mimic of an injury signal is a heat shock protein.

Preferred methods include embodiments wherein said mimic of an injury signal is hsp90.

Preferred methods include embodiments wherein said mimic of an injury signal is hsp60.

Preferred methods include embodiments wherein said mimic of an injury signal is bacterial cell wall extract.

Preferred methods include embodiments wherein said mimic of an injury signal is zymosan.

Preferred methods include embodiments wherein said mimic of an injury signal is interferon gamma.

Preferred methods include embodiments wherein said mimic of an injury signal is interferon alpha.

### DETAILED DESCRIPTION OF THE INVENTION

The invention teaches generation of dendritic cell-mesenchymal cell chimeras from iPSC cells, as well as gene modification/gene editing of the iPSC to endow enhanced therapeutic properties to iPSC derived mesenchymal/mesenchymal-like cells.

A “dendritic cell” (DC) is an antigen presenting cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. The DC has a characteristic morphology with thin sheets (lamellipodia) extending in multiple directions away from the dendritic cell body. Typically, dendritic cells express high levels of MHC and costimulatory (e.g., B7-1 and B7-2) molecules. Dendritic cells can induce antigen specific differentiation of T cells in vitro, and are able to initiate primary T cell responses in vitro and in vivo. Dendritic cells and T cells develop from hematopoietic stem cells along divergent “differentiation pathways.” A differentiation pathway describes a series of cellular transformations undergone by developing cells in a specific lineage. T cells differentiate from lymphopoietic precursors, whereas DC differentiate from precursors of the monocytemacrophage lineage.

“Cytokines” are protein or glycoprotein signaling molecules involved in the regulation of cellular proliferation and differentiation. Cytokines involved in differentiation and regulation of cells of the immune system include various structurally related or unrelated lymphokines (e.g., granulocyte-macrophage colony stimulating factor (GM-CSF), interferons (IFNs)) and interleukins (IL-1, IL-2, etc.)

A “polynucleotide sequence” is a nucleic acid (which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

An “amino acid sequence” is a polymer of amino acids (a protein, polypeptide, etc.) or a character string representing an amino acid polymer, depending on context. Either the given nucleic acid or the complementary nucleic acid can be determined from any specified polynucleotide sequence.

An “antigen” is a substance which can induce an immune response in a host or subject, such as a mammal. Such an antigenic substance is typically capable of eliciting the formation of antibodies in a host or subject or generating a specific population of lymphocytes reactive with that substance. Antigens are typically macromolecules (e.g., proteins, peptides, or fragments thereof; polysaccharides or fragments thereof) that are foreign to the host. A protein antigen or peptide antigen, or fragment thereof may be termed “antigenic protein” or “antigenic peptide,” respectively.” A fragment of an antigen is termed an “antigenic fragment.” An antigenic fragment has antigenic properties and can induce an immune response as described above.

An “immunogen” refers to a substance that is capable of provoking an immune response. Examples of immunogens include, e.g., antigens, autoantigens that play a role in induction of autoimmune diseases, and tumor-associated antigens expressed on cancer cells.

The term “immunoassay” includes an assay that uses an antibody or immunogen to bind or specifically bind an antigen. The immunoassay is typically characterized by the use of specific binding properties of a particular antibody to isolate, target, and/or quantify the antigen.

A vector is a composition or component for facilitating cell transduction by a selected nucleic acid, or expression of the nucleic acid in the cell. Vectors include, e.g., plasmids, cosmids, viruses, YACs, bacteria, poly-lysine, etc. An “expression vector” is a nucleic acid construct, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen. The term “enhanced function of a T cell”, “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

“Mesenchymal stem cells” (MSC) as used herein refers to multipotent stem cells with self-renewal capacity and the ability to differentiate into osteoblasts, chondrocytes, and adipocytes, among other mesenchymal cell lineages. In addition to these characteristics, MSCs may be identified by the expression of one or more markers as further described herein. Such cells may be used to treat a range of clinical conditions, including immunological disorders as well as degenerative diseases such as graft-versus-host disease (GVHD), myocardial infarction and inflammatory and autoimmune diseases and disorders, among others. Except where the context indicates otherwise, MSCs may include cells from adult sources and cord blood. MSCs (or a cell from which they are generated, such as a pluripotent cell) may be genetically modified or otherwise modified to increase longevity, potency, homing, or to deliver a desired factor in the MSCs or cells that are differentiated from such MSCs. As non-limiting examples thereof, the MSCs cells may be genetically modified to express Sirtl (thereby increasing longevity), express one or more telomerase subunit genes optionally under the control of an inducible or repressible promoter, incorporate a fluorescent label, incorporate iron oxide particles or other such reagent (which could be used for cell tracking via in vivo imaging, MRI, etc., see Thu et al., Nat Med. 2012 Feb. 26; 18(3):463-7), express bFGF which may improve longevity (see Go et al., J. Biochem. 142, 741-748 (2007)), express CXCR4 for homing (see Shi et al., Haematologica. 2007 July; 92(7):897-904), express recombinant TRAIL to induce caspase-mediatedx apoptosis in cancer cells like Gliomas (see Sasportas et al., Proc Natl Acad Sci USA. 2009 Mar. 24; 106(12):4822-7), etc.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization, “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

It is recognized that induced pluripotent stem cells (iPS cells) are virtually identical to ES cells at molecular and functional levels, there are critical hurdles to translation of their therapeutic potentials into medical applications. One of the issues is that because the current standard protocols for reprogramming and propagation of iPS cells include animal-derived materials that are unsuitable for potential clinical purposes, a fully defined method to generate and expand hiPS cells needs to be developed. For the purposes of the current invention induced pluripotent stem cells (iPS) are described by Shinya Yamanaka's team at Kyoto University, Japan. Yamanaka identified genes that are particularly active in embryonic stem cells, and used retroviruses to transfect mouse fibroblasts with a selection of those genes. Eventually, four key pluripotency genes essential for the production of pluripotent stem cells were isolated; Oct-3/4, SOX2, c-Myc, and Klf4. Cells were isolated by antibiotic selection for Fbx15+ cells. The same group published a study along with two other independent research groups from Harvard, MIT, and the University of California, Los Angeles, showing successful reprogramming of mouse fibroblasts into iPS and even producing a viable chimera.

The generation of human iPS cells by retroviral expression of four reprogramming factors (RFs; also referred to a de-differentiation factors) opened the potential for regenerative medicine therapies based on patient-specific, personalized stem cells. However, the insertional mutagenic potential of retroviruses combined with the potential for latent RF gene activation, especially c-MYC, all but eliminates integrative DNA-based approaches for use in regenerative medicine therapies. Other DNA-based iPS approaches using episomal vectors, adenovirus, integrated and excised piggyBac transposon or floxed lentivirus have been developed; however, these approaches either suffer from low efficiency of iPS cell generation or require genomic excision strategies that leaves behind an inserted DNA element tag. RNA-based iPS cell approaches using Sendai virus or mRNA transfection avoid potential integration problems associated with DNA-based approaches and are inherently safer methods for clinical applications. Although Sendai virus offers a reasonably efficient iPS approach, problems associated with persistent Sendai virus replication in iPS cell clones requires a negative selection step followed by several recloning steps from the single cell level to isolate virus-free iPS cells, such processes result in excessive iPS cellular division and passage. One of the more promising non-DNA based approaches involves daily transfection of four individual RF mRNAs (plus GFP mRNA) over 16 days. Unfortunately, this approach remains problematic. For example, experiments to replace KLF4 and retroviruses with corresponding transfected mRNAs were performed and the results validated; however OCT4 and SOX2 retroviruses could not be replaced with transfected mRNAs. The problem appears to stem from both the rapid degradation of RF mRNAs combined with the inconsistent cell-to-cell threshold expression level variation over time, which derives from attempting to transfect four independent mRNAs into the same cell on a daily basis for >14 days during reprogramming. Consequently, there remains a significant need for a simple and highly reproducible, non-DNA based approach to generate human iPS cells.

The disclosure provides methods and compositions for generating iPS cells from somatic cells (e.g., fibroblast cells, stromal cells, mesenchymal stem cells, and other somatic tissues). In some embodiments, iPSC are used to generate mesenchymal stromal cells are positive for (i) at least one of CD10, CD24, IL-11, AIRE-1, ANG-1, CXCL1, CD015, CD73 and CD90; (ii) at least one of CD10, CD24, IL-11, AIRE-1, ANG-1, CXCL1, CD105, CD73, CD90, CD105, CD13. CD29, CD44, CD166, CD274, and HLA-ABC; or (iii) any combination thereof. In another aspect, at least 50% of said mesenchymal stromal cells are positive for (i) at least two of CD10, CD24, IL-1, AIRE-1, ANG-1, CXCL1, CD105, CD73 and CD90; (ii) all of CD10, CD24, IL-11, AIRE-1, ANG-1, CXCL1, CD105, CD73, CD90, CD105, CD13, CD29, CD44, CD166, CD274, and HLA-ABC. In yet another aspect, at least 50% of said mesenchymal stromal cells are (i) positive for all of CD10, CD24, IL-11, AIRE-1, ANG-1, CXCL1, CD105, CD73, CD90, CD105. CD13, CD29, CD44, CD166, CD274, and HLA-ABC and (ii) do not express or express low levels of at least one of CD31, 34, 45, 133, FGFR2, CD271, Stro-1, CXCR4, TLR3. Additionally, at least 60%, 70%, 80% or 90% of such mesenchymal stromal cells may be positive for (i) one or more of CD10, CD24, IL-11, AIRE-1, ANG-1, CXCL1, CD105, CD73 and CD90; or (ii) one or more of CD10, CD24, IL-11, AIRE-1, ANG-1. CXCL1. CD105, CD73, CD90, CD105, CD13, CD29, CD44, CD166, CD274, and -HLA-ABC.

In one embodiment, the invention teaches the generation of pharmaceutical preparation comprises an amount of mesenchymal stromal cells effective to treat or prevent an unwanted immune response in a subject in need thereof. The pharmaceutical preparation may further comprise other cells, tissues or organs for transplantation into a recipient in need thereof. Exemplary other cells or tissues include RPE cells, skin cells, corneal cells, pancreatic cells, liver cells, or cardiac cells or tissue containing any of said cells. In another aspect, the pharmaceutical preparation comprises an amount of mesenchymal stromal cells effective to treat or prevent pain, heat sensitivity, and/or cold sensitivity.

In one embodiment of the, the mesenchymal stem cells derived from pluripotent stem cells are transfected with the IL-27 gene. The cells according to the invention may be cultured in a medium comprising .alpha.MEM. In another aspect, the mesenchymal stem cells derived from pluripotent stem cells may be cultured in a medium comprising serum or a serum replacement. For example, the mesenchymal stem cells derived from pluripotent stem cells cells may be cultured in a medium comprising, .alpha.MEM supplemented with 0%, 1%, 2%, 3%, 4%, 5%, 6%, 7%, 8%, 9%, 10%, 11%, 12%, 13%, 14%, 15%, 16%, 17%, 18%, 19%, or 20% fetal calf serum. In additional exemplary embodiments the medium may comprise higher percentages of fetal calf serum, e.g., more than 20%, e.g., at least 25%, at least 30%, at least 35%, at least 40%, or even higher percentages of fetal calf serum. The mesenchymal stem cells derived from pluripotent stem cells may be cultured on said matrix for at least about 14, 15, 16, 17, 18, 19, 20, 21, 22, 23, 24, 26, 27, 28, 29, or 30 days. In embodiment aspect, the mesenchymal stem cells derived from pluripotent stem cells are differentiated from pluripotent cells, e.g., iPS cells, or blastomeres. The pluripotent cells may be derived from one or more blastomeres without the destruction of a human embryo. Additionally, the mesenchymal stem cells derived from pluripotent stem cells may be differentiated from pluripotent cells by a method comprising (a) culturing said pluripotent cells to form clusters of cells. In one aspect, the pluripotent cells are cultured in the presence of vascular endothelial growth factor (VEGF) and/or bone morphogenic protein 4 (BMP-4). VEGF and BMP-4 may be added to the pluripotent cell culture within 0-48 hours of initiation of said cell culture, and said VEGF is optionally added at a concentration of 20-100 nm/mL and said BMP-4 is optionally added at a concentration of 15-100 ng/mL. In one aspect, the mesenchymal stem cells derived from pluripotent stem cells are differentiated from pluripotent cells by a method further comprising: (b) culturing said single cells in the presence of at least one growth factor in an amount sufficient to induce the differentiation of said clusters of cells into mesenchymal stem cells derived from pluripotent stem cells. The at least one growth factor added in step (b) may comprise one or more of basic fibroblast growth factor (bFGF), vascular endothelial growth factor (VEGF), bone morphogenic protein 4 (BMP-4), stem cell factor (SCF), Flt 3L (FL), thrombopoietin (TPO), EPO, and/or tPTD-HOXB4. The one or more of said at least one growth factor added in step (b) may be added to said culture within 36-60 hours from the start of step (a). Preferably, the one or more of said at least one growth factor added in step (b) is added to said culture within 40-48 hours from the start of step (a). The at least one factor added in step (b) may comprise one or more of bFGF, VEGF, BMP-4, SCF, FL and/or tPTD-HOXB4. The concentration of said growth factors if added in step (b) may range from about the following: bFGF is is about 20-25 ng/ml, VEGF is about 20-100 ng/ml, BMP-4 is about 15-100 ng/ml, SCF is about 20-50 ng/ml, FL is about 10-50 ng/ml, TPO is about 20-50 ng/ml, and tPTD-HOXB4 is about 1.5-5 U/ml.

In one aspect, the mesenchymal stromal cells of the present invention (a) do not clump or clump substantially less than mesenchymal stromal cells derived directly from ESCs; (b) more easily disperse when splitting compared to mesenchymal stromal cells derived directly from ESCs; (c) are greater in number than mesenchymal stromal cells derived directly from ESCs when starting with equivalent numbers of ESCs; and/or (d) acquire characteristic mesenchymal cell surface markers earlier than mesenchymal stromal cells derived directly from ESCs. The invention contemplates a pharmaceutical preparation comprising such mesenchymal stromal cells, which comprises an amount of mesenchymal stromal cells effective to treat an unwanted immune response. The invention also contemplates a pharmaceutical preparation comprising such mesenchymal stromal cells, which comprises an amount of mesenchymal stromal cells effective to treat or prevent pain, heat sensitivity, and/or cold sensitivity. The preparation may comprise an amount of mesenchymal stromal cells effective to treat an unwanted immune response and may further comprise other cells or tissues for transplantation into a recipient in need thereof. Exemplary other cells include allogeneic or syngeneic pancreatic, neural, liver, RPE, or corneal cells or tissues containing any of the foregoing. The pharmaceutical preparation may be useful in treating an autoimmune disorder or an immune reaction against allogeneic cells including, but not limited to, multiple sclerosis, systemic sclerosis, hematological malignancies, myocardial infarction, organ transplantation rejection, chronic allograft nephropathy, cirrhosis, liver failure, heart failure, GvHD, tibial fracture, bone factures, left ventricular dysfunction, leukemia, myelodysplastic syndrome, Crohn's disease, diabetes, obesity, metabolic diseases and disorders (such as lysosomal storage diseases including Tay-Sachs disease, Gaucher disease, Pompe disease, Hurler syndrome and metachromatic leukodystrophy), fatty liver disease, chronic obstructive pulmonary disease, osteogenesis imperfecta, homozygous familial hypocholesterolemia, hypocholesterolemia, treatment following meniscectomy, adult periodontitis, periodontitis, vasculogenesis in patients with severe myocardial ischemia, spinal cord injury, osteodysplasia, critical limb ischemia, diabetic foot disease, primary Sjogren's syndrome, osteoarthritis, cartilage defects, laminitis, multisystem atrophy, amyotropic lateral sclerosis, cardiac surgery, systemic lupus erythematosis, living kidney allografts, nonmalignant red blood cell disorders, thermal burn, radiation burn, Parkinson's disease, microfractures, epidermolysis bullosa, severe coronary ischemia, idiopathic dilated cardiomyopathy, osteonecrosis femoral head, lupus nephritis, bone void defects, ischemic cerebral stroke, after stroke, acute radiation syndrome, pulmonary disease, arthritis, bone regeneration, uveitis or combinations thereof. The MSCs of the invention (including formulations or preparations thereof) may be used to treat respiratory conditions, particularly those including inflammatory components or acute injury, such as Adult Respiratory Distress Syndrome, post-traumatic Adult Respiratory Distress Syndrome, transplant lung disease, Chronic Obstructive Pulmonary

Disease, emphysema, chronic obstructive bronchitis, bronchitis, an allergic reaction, damage due to bacterial or viral pneumonia, asthma, exposure to irritants, and tobacco use. Additionally, the MSCs of the invention (including formulations or preparations thereof) may be used to treat atopic dermatitis, allergic rhinitis, hearing loss (particularly autoimmune hearing loss or noise-induced hearing loss), psoriasis. Additionally, the subject MSC (including formulations or preparations thereof) may be useful to treat or prevent pain, heat sensitivity, and/or cold sensitivity.

1. A method of treating a degenerative condition comprising the steps of: a) obtaining a pluripotent stem cell; b) inducing expression in said pluripotent stem cell of one or more factors capable of promoting differentiation into desired cell lineage; c) differentiating said stem cell into a regenerative cell; d) optionally endowing said differentiated regenerative cell with chemoattractant activity; and e) optionally endowing said regenerative cell with immune modulatory activity.

2. The method of claim 1, wherein said degenerative condition is associated with loss of function of one more organ systems.

3. The method of claim 1, wherein said degenerative condition is associated with deterioration of function of one more organ systems.

4. The method of claim 1, wherein said degenerative condition is associated with fibrosis one more organ systems.

5. The method of claim 1, wherein said degenerative condition is associated with inflammation occurring in one more organ systems.

6. The method of claim 1, wherein said degenerative condition is associated with autoimmune attack against one more organ systems.

7. The method of claim 1, wherein said degenerative condition is associated with denervation of one more organ systems.

8. The method of claim 1, wherein said degenerative condition is associated with fibrosis one more organ systems.

9. The method of claim 2, wherein said organ systems are chosen from a group comprising of: a) respiratory system; b) digestive and excretory system; c) circulatory system; d) urinary system; e) integumentary system; f) skeletal system; g) muscular system h) endocrine system; i) lymphatic system; j) nervous system; and k) reproductive systems.

10. The method of claim 1, wherein said pluripotent stem cell is an inducible pluripotent stem cell.

11. The method of claim 10, wherein said pluripotent stem cell is generated by dedifferentiation.

12. The method of claim 11, wherein said dedifferentiation is accomplished by introduction into cells proteins capable of inducing dedifferentiation.

13. The method of claim 12, wherein said dedifferentiation results in cells expression pluripotency markers.

14. The method of claim 13, wherein said pluripotency marker is TRA-1-60.

15. The method of claim 13, wherein said pluripotency marker is hTERT.

16. The method of claim 13, wherein said pluripotency marker is TRA-1-81.

17. The method of claim 13, wherein said pluripotency marker is SSEA4.

18. The method of claim 13, wherein said pluripotency marker is OCT4.

19. The method of claim 13, wherein said pluripotency marker is SOX2.

20. The method of claim 13, wherein said proteins capable of inducing dedifferentiation are selected from a group comprising of: a) OCT4; b) NANOG; c) KLF-1; d) SOX-2; and e) h-RAS.
